(19) United States
(12) Patent Application Publication
(10) Pub. No.: US 2025/0241825 A1
(43) Pub. Date: Jul. 31, 2025

(54) NEEDLE SUPPORT WITH INTEGRATED TUBING MANAGEMENT

(71) Applicant: Life Technologies Corporation, Carlsbad, CA (US)

(72) Inventors: Carly Ann Litton, Logan, UT (US); Logan Trimble Williams, North Logan, UT (US); Jacob Andrew Kreider, Dauphin, PA (US); Daniel Jay Gibson, Wellsvile, UT (US); Michael Derrick, Logan, UT (US); Derik R. West, Logan, UT (US); Kylie Spackman, Smithfield, UT (US)

(21) Appl. No.: 19/040,716

(22) Filed: Jan. 29, 2025

Related U.S. Application Data
(60) Provisional application No. 63/626,380, filed on Jan. 29, 2024.

(51) Int. Cl. A61J 1/20

(52) U.S. Cl. CPC A61J 1/20

(57) ABSTRACT

Described herein are various embodiments and related methods of a needle and tubing organizing support system that can include one or more of a tray assembly configured to protect and retain a portion of a needle assembly and a securing mechanism. The tray assembly can include a tray body and a capturing recess extending along the tray body for at least partially containing a needle of the needle assembly. The tray assembly can further include a retention element and/or a sheath shaped to extend along a length of the capturing recess and encompass at least a portion of the needle of the needle assembly. The securing mechanism can be coupled to the tray body and configured to releasably secure a tubing of the needle assembly to the tray body.

### CROSS-REFERENCE TO RELATED APPLICATIONS

This application claims the benefit of the U.S. Provisional Patent Application Ser. No. 63/626,380 filed Jan. 29, 2024, and titled “NEEDLE SUPPORT WITH INTEGRATED TUBING MANAGEMENT,” which is incorporated herein by specific reference.

### TECHNICAL FIELD

The subject matter described herein relates generally to a needle support, including integrated tubing management, and more specifically to a needle and tubing organizing support system for needle assemblies, including fill needles for fill and finish assemblies.

### BACKGROUND

A process of filling vials with a pharmaceutical product, such as a vaccine or drug product, and finishing the process of packaging the medicine for distribution is commonly referred to in the art as “fill and finish”. The fill and finish process can be a common bottleneck in the manufacturing and deployment of vaccines due to, for example, insufficient scalability and/or lengthy set-up protocols.

For example, the filling process can take place within an isolator in order to maintain sterility of the pharmaceutical product. Multiple vials can be filled at once in the isolator using multiple needles, with each needle connected to a pump, surge bag, or feed line. Current devices for protecting needles and managing tubing can be difficult to manipulate with one hand and can fail to provide sufficient protection of the needle. Aspects described herein relate generally to needle and sheath organizing supports with integrated tubing management for fill needles that address deficiencies in currently available devices.

### SUMMARY

Systems, methods, and articles of manufacture are provided for needle organizing support, including integrated tubing management. The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

In an aspect of the present disclosure, a system for needle and tubing organization is provided. The system includes a tray assembly configured to protect and retain a portion of a needle assembly. The tray assembly includes a tray body including at least a portion of the tray body extending along a first plane, a capturing recess extending along the tray body and forming an opening along the tray body, the capturing recess including a capturing wall spanning across the opening and protruding away from the tray body and the first plane. The capturing recess is configured to at least partially contain a needle of the needle assembly, a retention element comprising a first retention portion and a second retention portion, the first retention portion extending between a proximal first retention end and a distal first retention end, the proximal first retention end positioned along the tray body, the distal first retention end being offset from the tray body and the first plane, the second retention portion extending towards the capturing recess from the distal first retention end; and a sheath shaped to extend along a length of the capturing recess and configured to encompass at least a portion of the needle of the needle assembly, at least a portion of the sheath configured to be releasably captured by the second retention portion and the tray body to assist with securing a position of the sheath relative to the capturing recess; and a securing mechanism coupled to the tray body and configured to releasably secure a tubing of the needle assembly to the tray body.

In another aspect of the present disclosure, a needle and tubing organizing support kit is provided. The kit includes a needle assembly, including a fill needle; and a tubing coupled at a first end to the fill needle; and a needle and tubing organizing support system. The needle and tubing organizing support system includes a tray assembly configured to protect and retain a portion of the needle assembly. The tray assembly includes a tray body, at least a portion of the tray body extending along a first plane; a capturing recess extending along the tray body and forming an opening along the tray body, the capturing recess comprising a capturing wall spanning across the opening and protruding away from the tray body and the first plane, and the capturing recess being configured to at least partially contain the fill needle of the needle assembly; a retention element including a first retention portion and a second retention portion, the first retention portion extending between a proximal first retention end and a distal first retention end, the proximal first retention end positioned along the tray body, the distal first retention end being offset from the tray body and the first plane, the second retention portion extending towards the capturing recess from the distal first retention end; and a sheath shaped to extend along a length of the capturing recess and configured to encompass at least a portion of the fill needle of the needle assembly, at least a portion of the sheath configured to be releasably captured by the second retention portion and the tray body to assist with securing a position of the sheath relative to the capturing recess; and a securing mechanism coupled to the tray body and configured to releasably secure a tubing of the needle assembly to the tray body.

In another aspect of the disclosure, a tray assembly is provided. The tray assembly is configured to protect and retain a portion of a needle assembly, and the tray assembly includes a tray body, at least a portion of the tray body extending along a first plane, a capturing recess extending along the tray body and forming an opening along the tray body, the capturing recess including a capturing wall spanning across the opening and protruding away from the tray body and the first plane, the capturing recess being configured to at least partially contain a needle of the needle assembly, and a retention element configured to assist with securing a needle of the needle assembly within at least a part of the capturing recess.

In another aspect of the disclosure, a method for a needle and tubing organizing support system is provided. The method includes inserting a needle into a sheath, and coupling the sheath to a tray body of the needle and tubing organizing support system. The needle and tubing organizing support system can include a capturing recess extending along the tray body and forming an opening along the tray body, the capturing recess including a capturing wall spanning across the opening and protruding away from the tray body and the first plane, the capturing recess being configured to at least partially contain a needle of the needle assembly, and a retention element including a first retention portion and a second retention portion, the first retention portion extending between a proximal first retention end and a distal first retention end, the proximal first retention end positioned along the tray body, the distal first retention end being offset from the tray body and the first plane, the second retention portion extending towards the capturing recess from the distal first retention end, and the sheath being shaped to extend along a length of the capturing recess and configured to encompass at least a portion of the needle of the needle assembly, at least a portion of the sheath configured to be releasably captured by the second retention portion and the tray body to assist with securing a position of the sheath relative to the capturing recess.

In another aspect of the disclosure, a bag for a fill-and-finish assembly is provided. The bag includes a pliable housing comprising at least one panel and a perimeter seam defining a cavity within the pliable housing. Further, at least one inlet and outlet port extend from the pliable housing, and at least one corner seam extends from the perimeter seam.

In another aspect of the disclosure, a line set wrap for a fill and finish assembly is provided. The line set wrap includes a head portion and a body portion. The body portion includes at least two pouches, wherein each pouch is configured to receive a needle and tubing organizing support system, and the head portion is coupled to an upper end of the body portion.

In another aspect of the disclosure, a fill-and-finish assembly is provided. The fill and finish assembly includes a surge bag comprising at least one inlet port and one outlet port, a needle assembly connected to the inlet port and outlet port by tubing, and at least a line identifier secured to the tubing.

It is understood that each aspect of the present disclosure herein can include any of the features, options, systems components, method steps, and possibilities recited in association with any other aspect of the present disclosure or other embodiments set forth above or elsewhere within this application.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a front view of an embodiment of a tray assembly of an example needle and tubing organizing support system, consistent with implementations of the current subject matter;

FIG. 2 depicts a front view of the needle and tubing organizing support system of FIG. 1 including example embodiments of a needle and a sheath coupled to the tray assembly;

FIG. 3 depicts a front view of the needle and tubing organizing support system of FIG. 1 including an example needle, sheath, and securing mechanism;

FIG. 4 depicts a perspective view of another example needle and tubing organizing support system including an example fill needle, sheath, and securing mechanism, consistent with implementations of the current subject matter;

FIG. 5 depicts a front view of an embodiment of the tray assembly of the needle and tubing organizing support system of FIG. 4, consistent with implementations of the current subject matter;

FIG. 6 depicts a perspective view of the tray assembly of the needle and tubing organizing support system of FIG. 4, consistent with implementations of the current subject matter;

FIG. 7 depicts a front view of an embodiment of the tray assembly of the needle and tubing organizing support system of FIG. 4, consistent with implementations of the current subject matter;

FIG. 8 depicts a side view of the tray assembly of the needle and tubing organizing support system of FIG. 4, consistent with implementations of the current subject matter;

FIG. 9 depicts a cross sectional view of the tray assembly of the needle and tubing organizing support system of FIG. 4, taken along lines A-A in FIG. 8, consistent with implementations of the current subject matter;

FIG. 10 depicts a back view of the tray assembly of the needle and tubing organizing support system of FIG. 4, consistent with implementations of the current subject matter;

FIG. 11 depicts a front view of an embodiment of the tray assembly of another example needle and tubing organizing support system with an embodiment of the securing mechanism, consistent with implementations of the current subject matter;

FIG. 12 depicts a front view of an example fill needle, example sheath, and example tubing that can be used with an embodiment of the tray assembly of a needle and tubing organizing support system, consistent with implementations of the current subject matter;

FIG. 13 depicts a front view of another example fill needle, example sheath, and example tubing that can be used with an embodiment of the tray assembly of a needle and tubing organizing support system, consistent with implementations of the current subject matter;

FIG. 14 depicts a front view of the example fill needle, sheath, and tubing of FIG. 12 in use with the tray assembly of FIG. 11, consistent with implementations of the current subject matter;

FIG. 15 depicts a front view of the example needle, sheath, and tubing of FIG. 13 in use with the tray assembly of FIG. 11, consistent with implementations of the current subject matter;

FIG. 16 depicts a perspective view of another example needle and tubing organizing support system with securing mechanism attachment points, consistent with implementations of the current subject matter;

FIG. 17 depicts a perspective view of the needle and tubing organizing support system of FIG. 4, consistent with implementations of the current subject matter;

FIG. 18A depicts an example needle in use with a sheath, consistent with implementations of the current subject matter;

FIG. 18B depicts a cross sectional view of the example needle in use with an embodiment of the sheath of FIG. 18A;

FIG. 19A depicts another example needle in use with an embodiment of the sheath, consistent with implementations of the current subject matter;

FIG. 19B depicts a cross sectional view of the example needle in use with an embodiment of the sheath of FIG. 19A;

FIG. 20A depicts another example needle in use with an embodiment of the sheath, consistent with implementations of the current subject matter;

FIG. 20B depicts a cross sectional view of the example needle in use with an embodiment of the sheath of FIG. 20A;

FIG. 21A depicts another example needle in use with an embodiment of the sheath, consistent with implementations of the current subject matter;

FIG. 21B depicts a cross sectional view of the example needle in use with an embodiment of the sheath of FIG. 21A;

FIG. 22A depicts another example needle in use with an embodiment of the sheath, consistent with implementations of the current subject matter;

FIG. 22B depicts a cross sectional view of the example needle in use with an embodiment of the sheath of FIG. 22A;

FIG. 23A depicts a perspective of a tubing connector, consistent with implementations of the current subject matter;

FIG. 23B depicts a front view of the tubing connector of FIG. 23A, consistent with implementations of the current subject matter;

FIG. 24 depicts a perspective view of the tray assembly of the needle and tubing organizing support system of FIG. 1, consistent with implementations of the current subject matter;

FIG. 25 depicts a front view of the tray assembly of the needle and tubing organizing support system of FIG. 1, consistent with implementations of the current subject matter;

FIG. 26 is a side view of the tray assembly of the needle and tubing organizing support system of FIG. 1, consistent with implementations of the current subject matter;

FIG. 27 is a cross-sectional view of the tray assembly of the needle and tubing organizing support system of FIG. 1, taken along lines B-B in FIG. 26, consistent with implementations of the current subject matter;

FIG. 28 is a front view of the tray assembly and securement mechanism of the needle and tubing organizing support system of FIG. 1, consistent with implementations of the current subject matter;

FIG. 29 is a front view of the tray assembly, securement mechanism, needle, sheath, and tubing of the needle and tubing organizing support system of FIG. 1, consistent with implementations of the current subject matter;

FIG. 30 is a front view of the tray assembly, securement mechanism, needle, sheath, and tubing of the needle and tubing organizing support system of FIG. 1, consistent with implementations of the current subject matter;

FIG. 31 is a front view of the tray assembly, securement mechanism, needle, sheath, and tubing of the needle and tubing organizing support system of FIG. 1, consistent with implementations of the current subject matter;

FIG. 32 depicts a top perspective view of an example tray body of another embodiment of the needle and tubing organizing support system, consistent with implementations of the current subject matter;

FIG. 33 is a bottom perspective view of the tray body of FIG. 32, consistent with implementations of the current subject matter;

FIG. 34 depicts a front view of an example line set wrap that can be used with an embodiment of the tray assembly of a needle and tubing organizing support system, consistent with implementations of the current subject matter;

FIG. 35 depicts a flow chart for assembling a line set wrap, including the tray assembly of a needle and tubing organizing support system, consistent with implementations of the current subject matter;

FIG. 36 depicts a front view of an example surge bag that can be used with an embodiment of the tray assembly of a needle and tubing organizing support system, consistent with implementations of the current subject matter;

FIG. 37 illustrates a schematic of an example tubing line identifier/s coupled to a tubing line that can be used with an embodiment of the tray assembly of a needle and tubing organizing support system, consistent with implementations of the current subject matter;

FIG. 38A depicts a perspective view of an example tubing line identifier that can be used with an embodiment of the tray assembly of a needle and tubing organizing support system, consistent with implementations of the current subject matter;

FIG. 38B depicts a top view of an example tubing line identifier that can be used with an embodiment of the tray assembly of a needle and tubing organizing support system, consistent with implementations of the current subject matter;

FIG. 38C depicts a cross-sectional view of an example tubing line identifier of FIG. 38B, taken along lines AA of FIG. 38B, consistent with implementations of the current subject matter;

FIG. 39 illustrates a schematic of an example fill and finish system, consistent with implementations of the current subject matter; and

When practical, similar reference numbers denote similar structures, features, or elements.

### DETAILED DESCRIPTION

The fill and finish process can be an important step in the pharmaceutical manufacturing process. For example, the fill and finish process can include containers (e.g., vials, pouches, flexible containers) for receiving and containing one or more pharmaceutical products. Such pharmaceutical products can include, for example, one or more of a vaccine, biological and a drug substance product. The pharmaceutical products can include one or more biocomponents, including fluids, solids, mixtures, solutions, and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components, and the like. Examples of some common biological components include E. coli, yeast, bacillus, and CHO cells. Biological components can also comprise cell-therapy cultures and cells and microorganisms that are aerobic or anaerobic and adherent or non-adherent. The fill and finish process can also include packaging (e.g., safely packing and/or storing) the one or more pharmaceutical products for distribution. For example, the fill process can take place within an isolator (e.g., a clean air device that provides containment and separation of aseptic pharmaceutical processes from technical personnel and the surrounding work environment) and multiple containers can be filled at once using multiple filling devices (e.g., fill needles). Each filling device, for example, can be connected to a pump, surge bag, or feed line with its own fluid line (e.g., tubing). In other examples, each filling device with its fluid line can form a part of a fluid manifold assembly in fluid communication with a fluid sampling device connected to a surge bag. The filling devices (e.g., fill needles) and associated fluid lines must be aseptically packaged for placement into the isolator. In some instances, the fluid line can become tangled within the aseptic package. Additionally, the filling device and associated fluid line in each aseptic package are often required to be arranged and connected within an isolator using a single hand, and management/organization of filling devices and their respective fluid lines can be difficult to manipulate. Additionally, filling devices (e.g., fill needles) can be fragile and can bend or become damaged if left unprotected.

Consistent with implementations of the current subject matter, the needle and tubing organizing support system is configured to efficiently and effectively protect various embodiments of a filling device, such as a fill needle (e.g., a needle that can couple to a fill line (e.g., tubing) for dispensing one or more fluids from the fluid line into one or more containers). The needle and tubing organizing support system can also organize various embodiments of a fluid line, such as a variety of different sized and shaped tubing that can be permanently or releasably coupled to an embodiment of a fill needle. The needle and tubing organizing support system can enable efficient and effective use of the needle and tubing organizing support system, such as allowing single-handed use of the needle and tubing organizing support system and/or one or more elements that can be coupled and/or used with the needle and tubing organizing support system. In some instances, a user may only have one hand available to manipulate objects within the isolator. In some instances, a tubing should be released from a tray assembly without being dropped onto the lower surface of the isolator and becoming contaminated and/or otherwise unusable. For example, the needle and tubing organizing support system can be coupled to a surge bag, pump, or feed line using a user's single hand. This can allow a user to manipulate the fluid line or tubing of the needle and tubing organizing support system without the risk of dropping or contaminating the fluid line or tubing. Various embodiments of the needle and tubing organizing support system for securely organizing various embodiments of a needle assembly are described herein.

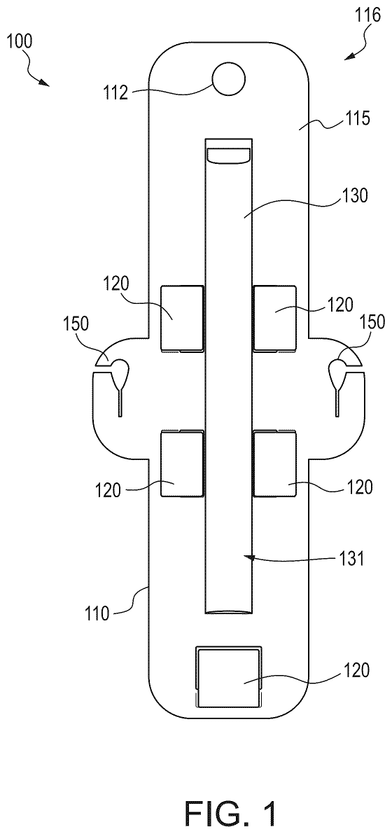

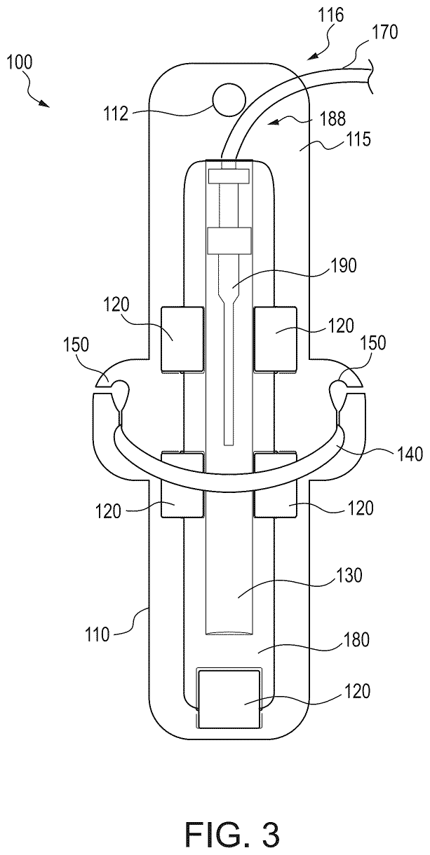

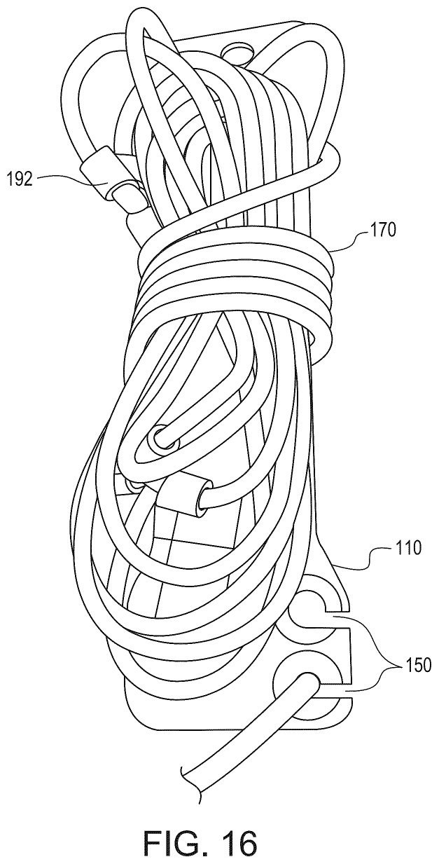

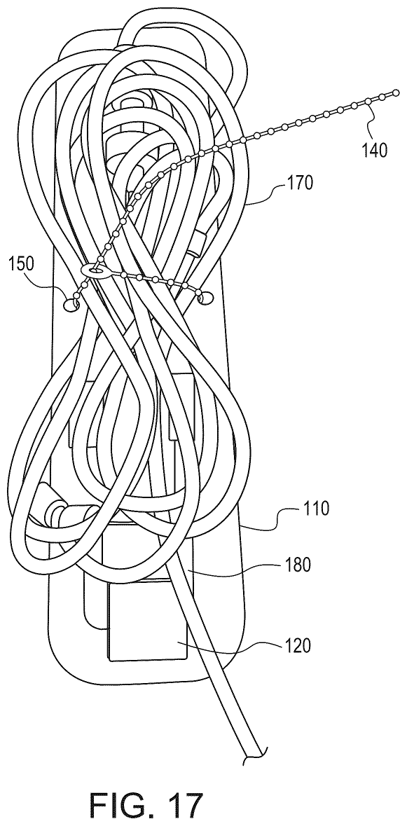

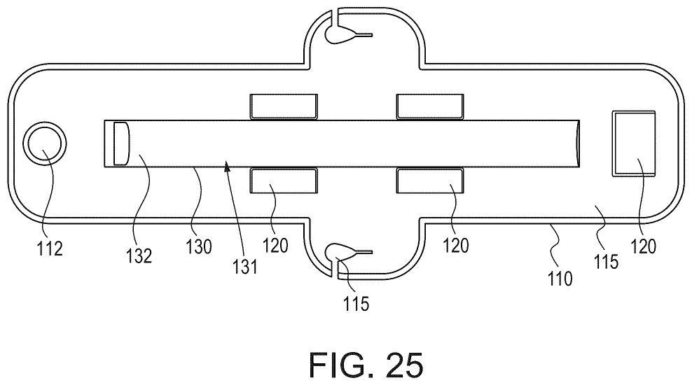

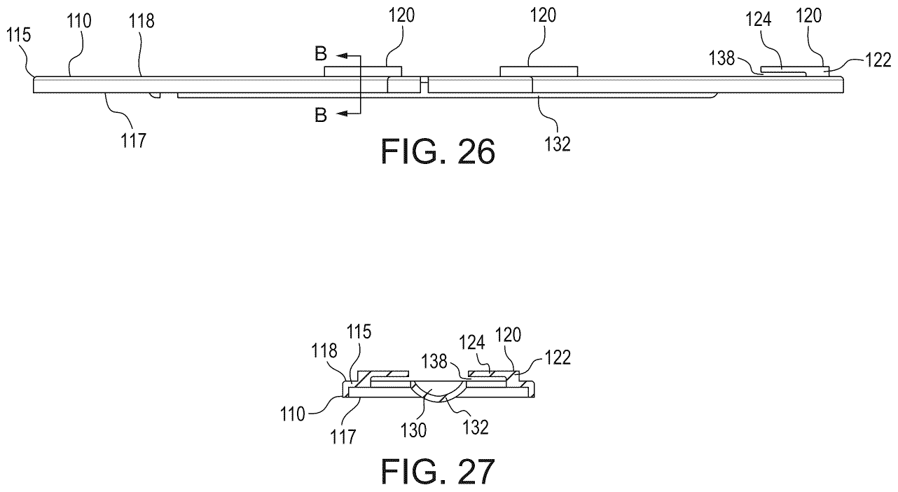

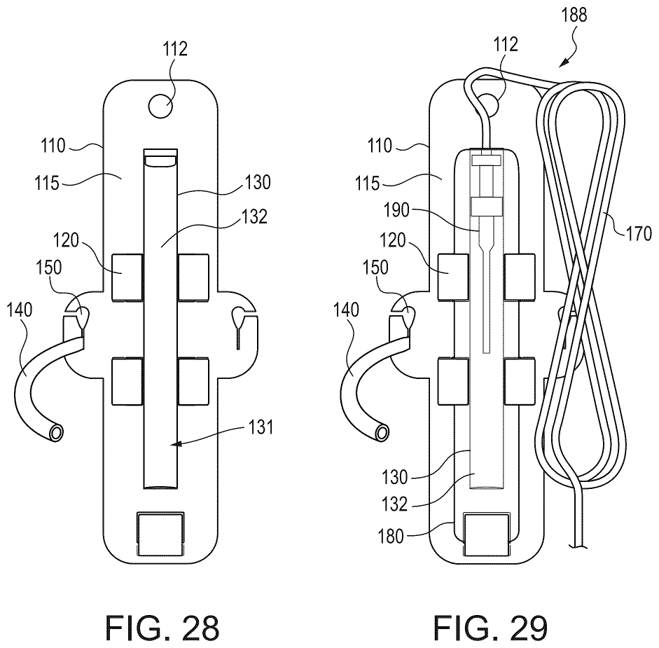

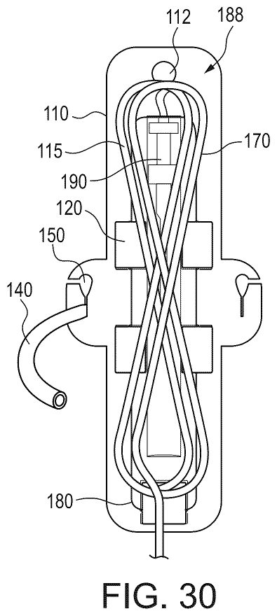

FIGS. 1-3 and 24-31 illustrate embodiments of a needle and tubing organizing support system 100 that is configured to protect and retain a portion of a needle assembly 188. For example, the needle assembly 188 can include a fill needle 190 and a length of tubing 170 coupled to the fill needle 190. As shown in FIGS. 1-3 and 24-31, the needle and tubing organizing support system 100 can include embodiments of a tray assembly 110 and a securing mechanism 140, as shown in FIGS. 3, 4, 16, 17, and 31 The tray assembly 110 can be configured to protect and retain a portion of an embodiment of the needle assembly 188, and the securing mechanism 140 can be configured to releasably secure the tubing of the needle assembly 188, as will be described in greater detail below. In some embodiments, the tray assembly 110 can include an integrated securing mechanism 140 such that the securing mechanism 140 forms a part of the tray assembly 110. In some embodiments, the securing mechanism 140 is coupled to the tray assembly 110, including releasably coupled to the tray assembly 110.

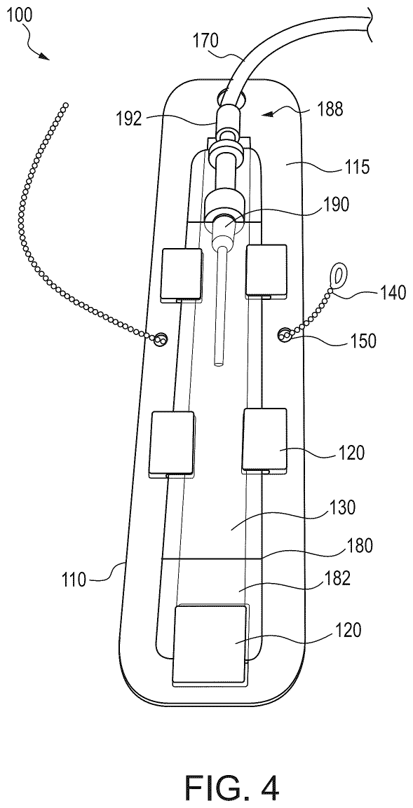

As shown in FIGS. 4 and 16, the connection between the tubing 170 and the fill needle 190 can be secured by a compression collar device 192. Extension of the tubing 170 to achieve a desired length for the fill and finish process can be achieved by connecting two more tubing 170 via fluid connecter secured with compression collar device 192. Examples of compression collar device 192 and how various tubings and fluid components can be coupled thereto for leak-proof fluid connections is disclosed in U.S. Pat No. 11,371,634 issued on Jun. 28, 2022, U.S. Pat No. 11,821,553 issued on Nov. 21, 2023, and U.S. Pat No. 11,821,554 issued on Nov. 21, 2023, which are incorporated herein by specific reference.

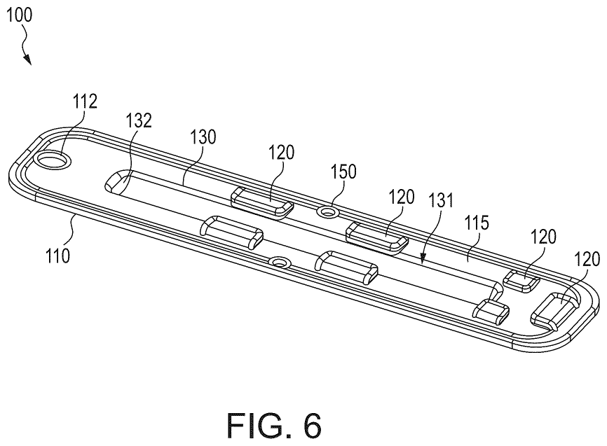

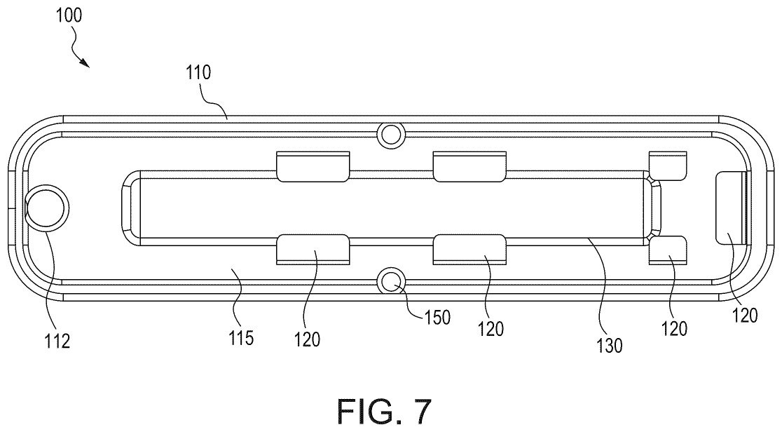

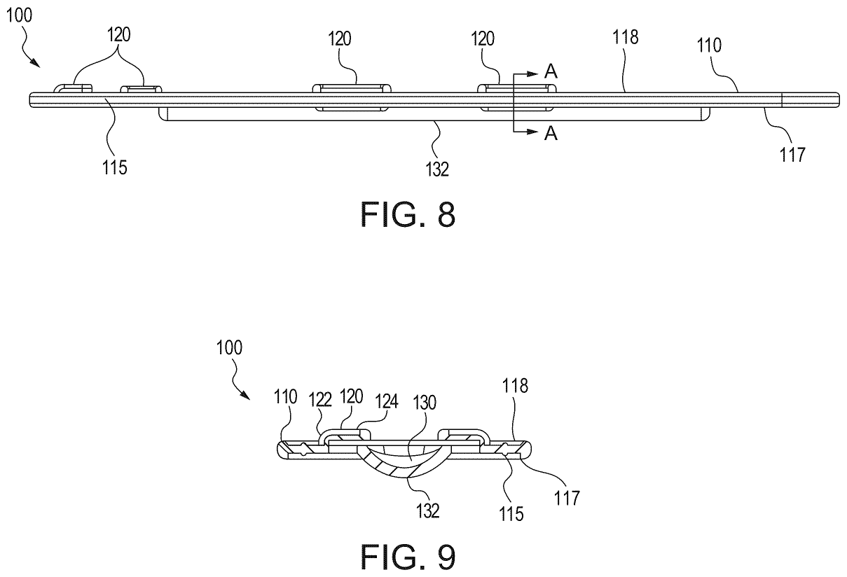

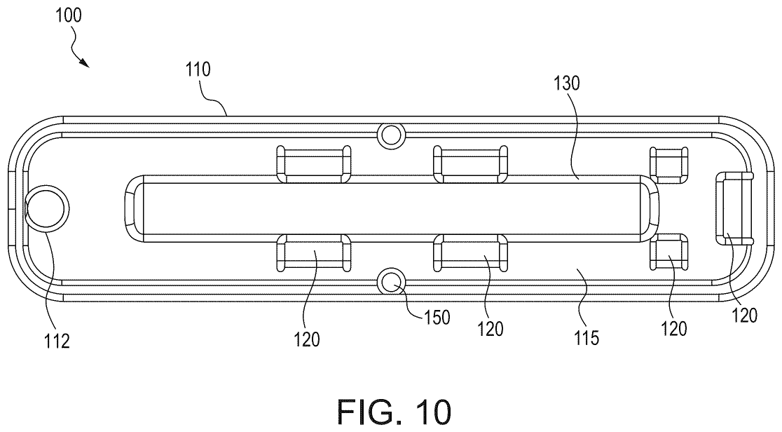

As shown in FIGS. 1-10, 24-29, 32 and 33 the tray assembly 110 can include a tray body 115, a capturing recess 130, and one or more retention elements 120. In some embodiments, the tray assembly 110 can also include a sheath 180 that is configured to encompass at least a portion of an embodiment of the fill needle 190 of the needle assembly 188. For example, an embodiment of the sheath 180 can include an elongated pocket that can receive and protect at least a part of the fill needle 190. In some embodiments, the needle and tubing organizing support system 100 can include a securing mechanism 140 and/or one or more securing mechanism attachment elements 150 that are configured to releasably secure an embodiment of the tubing 170 of the needle assembly 188 to the tray body 115, as will be described in greater detail below.

As shown in FIGS. 1-3 and 24-33, the tray body 115 can be formed of a single piece or multiple pieces coupled together, and can be formed out of one or more materials that are generally rigid. For example, the tray body 115 can be made of a rigid plastic and/or metal material. As shown in FIGS. 1-10 the tray body 115 can be generally rectangular in shape and can be substantially planar. Other shapes and configurations of the tray body 115 are within the scope of this disclosure. For example, at least a part of the tray body 115 can extend along a first plane and include an embodiment of a capturing recess 130 that is sized and shaped to assist with protecting and containing at least a part of an embodiment of the fill needle 190.

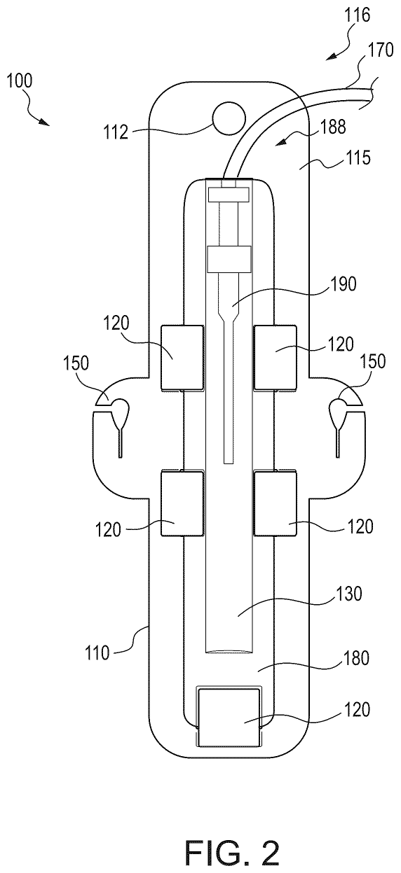

As shown in FIGS. 2-4 and 29-33 the capturing recess 130 can be configured to at least partially contain and/or surround an embodiment of the fill needle 190, such as an embodiment of the fill needle 190 that is positioned within a sheath 180 coupled to the tray body 115. As shown in FIG. 1, the capturing recess 130 can extend along the tray body 115, such as along and/or parallel to a longitudinal axis of the tray body 115. For example, the capturing recess 130 can form and/or include an opening 131 along the tray body 115 and along the first plane. The capturing recess 130 can include a capturing wall 132 defining at least a part of the capturing recess 130. For example, the capturing wall 132 can span across the opening 131 and protrude away from the tray body 115 and the first plane. As shown in FIGS. 1, 5, 6, 9, 10, 24, 25, 28, and 32-33 the capturing wall 132 can extend across and along the opening 131 in the tray body 115, as well as connect to one or more sides of the opening 131. At least a part of the capturing wall 132 can extend away from a bottom side 117 of the tray body 115, such as in a direction that is orthogonal to and/or non-parallel to the first plane. As such, the capturing wall 132 can define a volume of space within the capturing recess 130 for the protective sheath 180 and fill needle 190 to at least partially fit within.

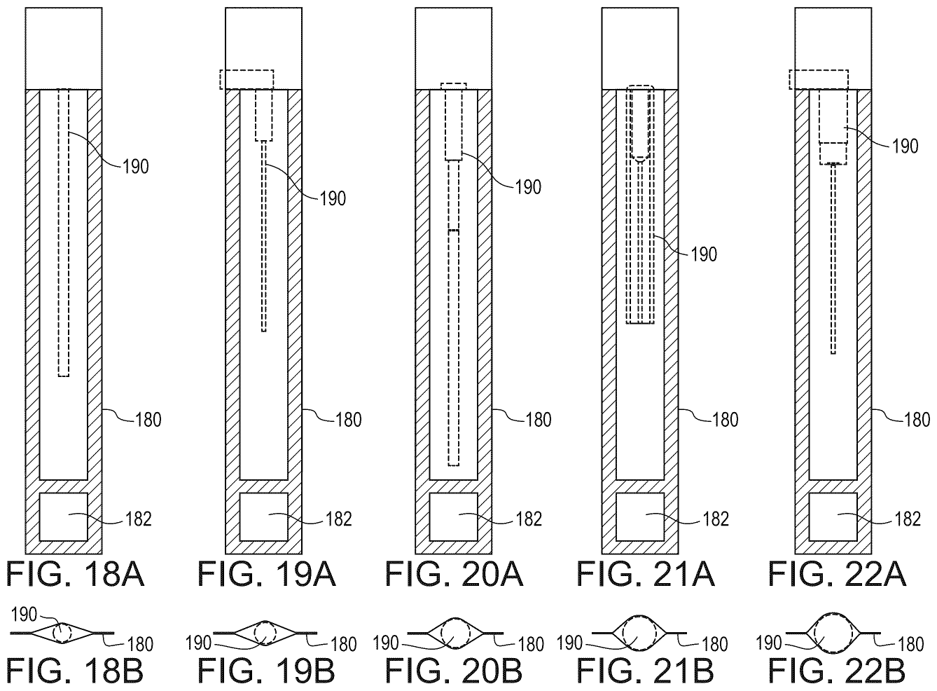

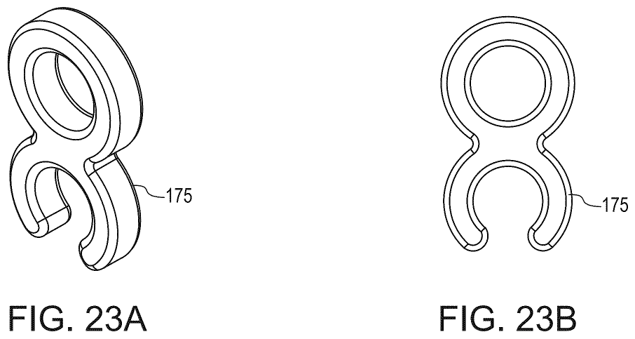

In some embodiments, the capturing recess 130 can be formed as a depression, groove, channel, or indentation in the tray body 115 defined by the capturing wall 132. As shown in FIGS. 9 and 27 the capturing wall 132, and the capturing recess 130 as a whole, can include a cross-section having a curved shape and/or U-shape. Other cross-sectional shapes of the capturing wall 132 and/or capturing recess 130 are within the scope of this disclosure, such as one or more of a square, flat, V-shaped, and rectangular shape. For example, a curved cross-section of the capturing wall 132 and capturing recess 130 can assist with protecting the fill needle 190 by increasing contact and/or surrounding area between the capturing wall 132 and the fill needle 190. For example, contact area between the capturing wall 132 and the fill needle 190 can distribute force loads (e.g., pressure) more evenly along the fill needle 190, while also allowing the capturing recess 130 to fit a variety of fill needles 190 having a variety of shapes and sizes. By at least partially containing and/or surrounding the protective sheath 180 containing the fill needle 190, the capturing recess 130 can assist in protecting the fill needle 190 from damage (e.g., bending of the fill needle 190) and act as a support for the fill needle 190. In some embodiments, the capturing recess 130 can be sized and shaped to receive a variety of fill needles 190 having a variety of shapes and sizes (e.g., varying diameters and/or lengths), as shown in FIGS. 18-22.

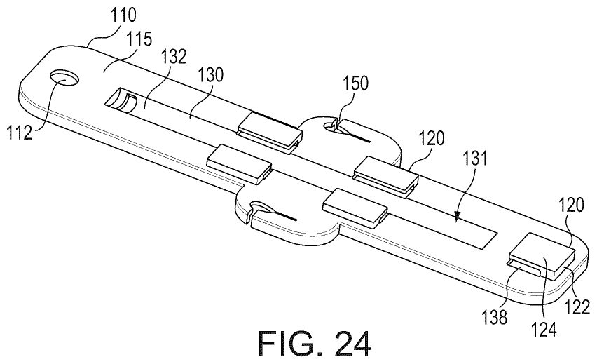

In some embodiments, the tray assembly 110 can further include one or more retention elements 120 formed along the tray body 115. For example, the retention elements 120 can be arranged around a perimeter of the capturing recess 130, such as along and/or adjacent to the opening 131. The retention elements 120 can be made of the same material as the tray body 115 or can be made of a different material (e.g., material that is more or less rigid). As explained in further detail below, retention elements 120 can assist with releasably securing an embodiment of the sheath 180 along the capturing recess 130, such as to secure and/or contain an embodiment of the fill needle 190 at least partly within the capturing recess 130. The retention elements 120 can include a variety of shapes and sizes for securing the protective sheath 180 along the capturing recess 130. For example, each retention element 120 can include one or more of a tab, a clip, and/or the like. As shown in FIGS. 1-3 and 24-33, the retention elements 120 can include at least one tab (e.g., extruding element) that protrudes away from a top side 118 of the tray body 115 and extends toward the capturing recess 130. As shown in FIGS. 24, 26, and 27, the retention element 120 can include a first retention portion 122 and a second retention portion 124. In some embodiments, the first retention portion 122 can extend between a proximal first retention end and a distal first retention end, where the proximal first retention end is positioned along the tray body 115 and the distal first retention end is offset from the tray body 115 and the first plane. The second retention portion 124 can extend toward the capturing recess 130 from the distal first retention end.

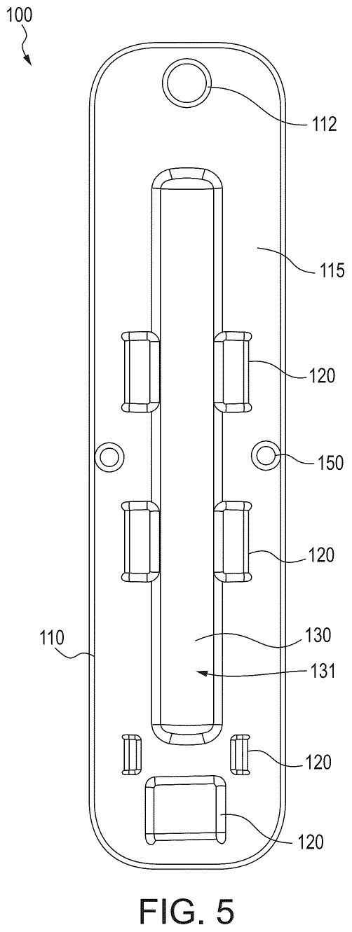

In some embodiments, the retention element 120 can be arranged to form a sheath receiving slot 138 between the retention element 120 and the tray body 115, as seen in FIGS. 24, 26, and 27. For example, the sheath 180 can be slid into this sheath receiving slot 138, as explained in further detail below. At least a portion of the sheath 180 can be held within the sheath receiving slot 138 to retain the fill needle 190 at least partially within the capturing recess 130. As shown in FIGS. 1-3 and 24-33, the needle and tubing organizing support system 100 can include five retention elements 120, including one retention element 120 along the bottom of the capturing recess 130 and two retention elements 120 on each side of the capturing recess 130, however, fewer or more retention elements 120 can be included. For example, as shown in FIGS. 5-10, the needle and tubing organizing support system 100 can include seven retention elements 120, including one retention element 120 along the bottom of the capturing recess 130 and three retention elements 120 on each side of the capturing recess 130. The retention elements 120 may be uniform in size and shape along the tray body 115, however, one or more retention elements 120 can differ in size and/or shape along the tray body 115. For example, the bottom retention element 120, as shown in FIG. 29, can assist with retaining the sheath 180 within and/or along the capturing recess 130, such as preventing the sheath 180 from slipping out of the capturing recess 130 when the needle and tubing organizing support system 100 is held upright. The retention elements 120 along either side of the capturing recess 130 can assist with retaining the sheath 180 within and/or along the capturing recess 130, such as preventing the sheath 180 from falling out of the capturing recess 130 when the needle and tubing organizing support system 100 is held various orientations (e.g., upside down).

For example, one or more of the tray body 115, capturing recess 130, and retention element 120 can be formed as a single part, such as molded as a single part. In some embodiments, at least one of the tray body 115, capturing recess 130, and retention element 120 can be formed as a separate part that is coupled together (e.g., the retention elements are coupled to the tray body during manufacturing). In some embodiments, one or more of the tray body 115, capturing recess 130, and retention element 120 can be made out of the same or similar material, such as when the tray body 115, capturing recess 130, and retention element 120 form a single part. In some embodiments, one or more of the tray body 115, capturing recess 130, and retention element 120 can be made out of different materials.

In some embodiments, the tray body 115 can include a length of 10 inches, 12 inches, 14 inches, and/or a length less than 16 inches. In some embodiments, the tray body 115 can have a length that is greater than 8 inches. The tray body 115 can include a rectangular shape and/or have one or more of a variety of shapes and sizes. The tray body 115 can be made out of one or more materials, such as a polypropylene, polycarbonate, polyethylene, polyethylene terephthalate, polyvinyl chloride, and polystyrene, and/or made of HDPE therefore withstanding both Gamma Irradiation and Autoclaving.

In some embodiments, the capturing recess 130 can include a length of 8 inches, 10 inches, 12 inches, 14 inches, and/or a length less than 16 inches. In some embodiments, the capturing recess 130 can have a length that is greater than 6 inches. The capturing recess 130 can include an elongated and rounded shape and/or have one or more of a variety of shapes and sizes. The capturing recess 130 can be made out of one or more materials, such as a polypropylene, polycarbonate, polyethylene, polyethylene terephthalate, polyvinyl chloride, and polystyrene, and/or made of HDPE therefore withstanding both Gamma Irradiation and Autoclaving.

In some embodiments, the retention element 120 can include one or more of a variety of shapes and sizes for capturing (including releasably capturing) a variety of sheaths. The retention element 120 can be made out of one or more materials, such as a polypropylene, polycarbonate, polyethylene, polyethylene terephthalate, polyvinyl chloride, and polystyrene, and/or made of HDPE therefore withstanding both Gamma Irradiation and Autoclaving.

The sheath 180 can be sized to fit a variety of fill needles 190. The sheath 180 can include longer or shorter sizes to fit fill needles 190 of varying length. For example, the sheath 180 can be made of a 3 layer film, such as Thermo Scientific™ CX3-9 film, with a seam along the two side edges and the bottom edge, with an opening at the top of the sheath 180, as shown in FIGS. 18-22. The sheath 180 can also include a seam adjacent to but a distance away from the bottom of the sheath 180, forming a protective gap 182 at the bottom of the sheath 180. The protective gap 182 can be enclosed by seams on all four sides, and provides a barrier between a bottom portion of the fill needle 190 and a bottom portion of the sheath 180. As such, the protective gap 182 can protect the bottom portion of the fill needle 190 from poking through the sheath 180 and/or hitting the bottom of the capturing recess 130, which could contaminate and/or damage the fill needle 190. For example, the sheath 180 can include a length of 4 inches, 5 inches, 6, inches, 7 inches, 8 inches, 9 inches, 10 inches, 11 inches, 12 inches, and/or a length less than 14 inches. In some embodiments, the sheath 180 can have a length that is greater than 3 inches. The sheath 180 can include a rectangular shape and/or have one or more of a variety of shapes and sizes. The sheath 180 can be made out of one or more materials, such as a polymer like polyester, polyethylene, polypropylene, polymethyl methacrylate, elastomers, a CX3-9 film, tie layer and nylon. The sheath 180 can be made of a material that is not easily punctured by the needle, thereby reducing the risk of particulate contaminating the pharmaceutical product being filled and increasing patient safety.

For example, the sheath 180 containing at least a portion of an embodiment of the fill needle 190 (of a needle assembly 188) can be placed into the capturing recess 130 of the tray assembly 110 by sliding the sheath 180 down and along the capturing recess 130 (e.g., from the top of the capturing recess 130) and into and along the sheath receiving slots 138 (e.g., between the tray body 115 and the retention elements 120). The sheath 180 can be removed by sliding the sheath 180 up and along the capturing recess 130 and receiving slots 138. In some embodiments, the sheath 180 can be removed by deforming and pulling the sheath 180 straight out of the capturing recess 130 and/or receiving slot 138, such as when the sheath 180 is formed of a flexible material and can deform to enable the sheath to be pulled away from the tray body 115 (e.g., pulled in a directed normal to the front side 118 of the tray body 115). In some embodiments, the sheath 180 can be made of a stiffer material that resists deflection and is limited to being slid up and out of the capturing recess 130 as opposed to being pulled straight out. Embodiments of the sheath 180 that are formed of a stiff material can help retain the sheath 180 and fill needle 190 at least partially within the capturing recess 130, which can help protect the fill needle 190 until removal of the sheath 180 and/or fill needle 190 by a user. The sheath 180 containing fill needle 190 is retained within the capturing recess 130 by the retention elements 120 even when the fill needle 190 and the needle and tubing organizing support system 100 is rotated in all three rotational directions (pitch, yaw, and roll) thus enabling the fill needle 190 and the needle and tubing organizing support system 100 to be a robust solution for needle protection and organization.

As shown in FIGS. 1-3, 5-7, 10-11, 14-15, 24-25, and 28-33, the tray assembly 110 can include a mounting element 112 to assist with mounting the needle and tubing organizing support system 100 to a fixture (e.g., hanger, hook, etc.), such as part of a setup step for performing a fill process. In some embodiments, the mounting element 112 can include a hole through the tray body 115 and can be located along a top portion 116 of the tray body 115, as shown in FIGS. 1-3, 5-7, 10-11. 14-15, 24-25, and 28-33. Other embodiments of the mounting element 112 can be included in the tray assembly 110, such as one or more of a hook, a hook and loop fabric, a magnet, and the like.

As shown in FIGS. 18-22, the protective sheath 180 and the capturing recess 130 can be sized to support a variety of needle sizes, including fill needles 190 with varying lengths and diameters. Different use cases may require different fill needles 190, and the universal size of the capturing recess 130 of the needle and tubing organizing support system 100 enables the needle and tubing organizing support system 100 to be used in a variety of use cases. For example, the needle 190 can include one or more of a 24 gauge needle or other suitable gauges. For example, the needle 190 can include an inner diameter of approximately 0.5 millimeter (mm) to approximately 7.0 mm. In some embodiments, the needle 190 can include a length of approximately 3 inches, approximately 4 inches, approximately 4.28 inches, approximately 4.41 inches, approximately 4.80 inches, approximately 5.25 inches, approximately 6.89 inches, and/or a length less than approximately 8.5 inches. In some embodiments, the needle 190 can have a length that is greater than 2.5 inches. The needle 190 can include a tubular shape and/or have one or more of a variety of profile and cross-sectional shapes. The needle 190 can be made out of one or more materials, such as a stainless steel, titanium, polyether ether ketone (PEEK), and niobium. In some embodiments, the needle 190 can include a coating (e.g., plastic coating).

Tubing 170 can be attached at one end to the fill needle 190, with another end loose for attachment to the surge bag, fill line, or pump. The length of tubing 170 can be unwieldy and benefits from organization and securement (e.g., during storage and/or before use) so that a user performing the fill and finish process can easily and quickly unpackage and use the elements of the needle and tubing organizing support system 100. For example, the tubing 170 can include one or more of a variety of tubing, such as tubing used in fill and finish processes. The tubing 170 can be made out of one or more materials (e.g., including materials having a variety of shore and hardness values), such as one or more of a silicone, a thermoplastic elastomer (TPE), and a polymer like polyvinylchloride, polyethylene, or polypropylene.

As shown in FIGS. 3, 4, 11, 14, 15, 17, and 28-31, some embodiments of the tray assembly 110 can include a securing mechanism 140. The securing mechanism 140 can be configured to releasably secure a tubing 170 of an embodiment of the needle assembly 188. For example, the securing mechanism 140 can keep tubing 170 untangled, protected, and organized. For example, the securing mechanism 140 can secure tubing 170 within an organized bundle, thereby preventing knots or kinks forming in the tubing, which can be damaging and interfere with the fill and finish process. As shown in FIGS. 1-3 and 24-31, the securing mechanism 140 can include a belt made of a separate piece of tubing. The securing mechanism 140 can extend across the middle of the tray body 115 to provide an even securement of the tubing 170 against the tray body 115. The securing mechanism 140 can be anchored or attached to the tray assembly 110 via securing mechanism attachment elements 150 located on the tray body 115. The securing attachment elements 150 may be located on wings or extensions of the tray body 115 as shown in FIGS. 1-3 and 24-31. The securing attachment elements 150 can include one or more slots, pinch-points, holes, and the like. The securing mechanism 140 can be used to organize, contain, or manage tubing 170 attached to the fill needle 190, as explained in further detail below.

The securing mechanism 140 can be a variety of suitable mechanisms. In some embodiments, the securing mechanism 140 can be a belt made of the tubing 170 attached to the needle (as shown in FIG. 16). In this embodiment, the securing attachment elements 150 may be located on the tray body 115 adjacent to a corner of the tray body 115, as shown in FIG. 16. The securing attachment elements 150 can include one or more slots, pinch-points (e.g., an opening or cutout with a narrowing diameter that can apply a compressive force to a securing mechanism to hold it in place), holes, and/or the like.

In some embodiments, the securing mechanism can be tubing 170 attached to the fill needle 190 and excess length wound around the tray body 115 and fill needle 190. A loose end of the tubing 170 can be secured to itself using a tubing connector 175 (i.e. a device that fixes in place a portion of tubing 170 relative to another portion of tubing 170), as seen in FIGS. 23A-23B. In some embodiments, the tubing connector 175 can be shaped generally like a figure-eight and can include an enclosed loop and an open loop. An end of tubing 170 can be inserted into the enclosed loop of connector 175, and another portion of tubing 170 can be removably inserted into the open loop of connector 175 to thereby releasably and safely secure the tubing 170 to itself.

In some embodiments, the securing mechanism 140 can be a beaded belt (e.g. an elongated belt or tie with a varying diameter to form beads that can be secured within a loop at the end of the elongated belt or tie), as shown in FIGS. 4, 11, 14, 15, and 17. The securing mechanism 140 can extend across the middle of the tray body 115 to provide a balanced securement of the tubing 170 against the tray body 115. The securing attachment elements 150 may be located adjacent to the capturing recess 130 as shown in FIGS. 4-11, 14, 15, and 17. The securing attachment elements 150 may include slots, pinch points, holes, and the like.

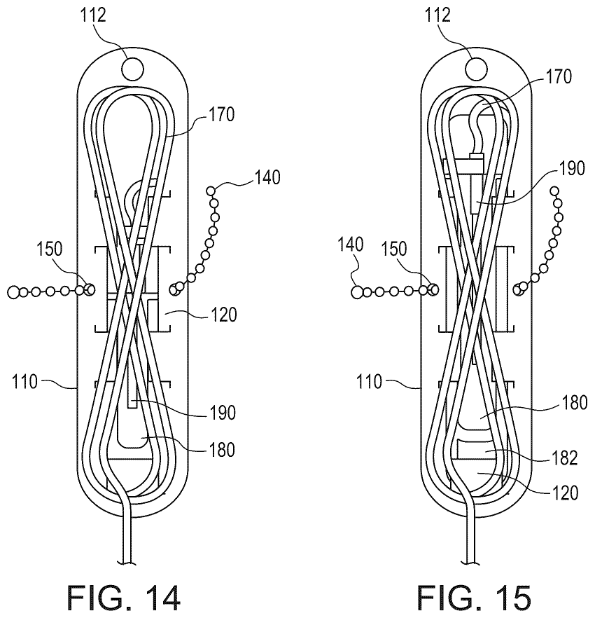

As mentioned above, the tray assembly 110 can include a securing mechanism 140 and securing attachment elements 150 used to secure the tubing 170 against the tray body 115 for transport or storage. For example, the tubing 170 can be wound and placed in front of the capturing recess 130, as shown in FIGS. 14-17 and 30-31. The securing mechanism 140, such as a beaded belt or a separate piece of tubing functioning as a belt, can be secured around the tubing 170 to hold the tubing 170 against the tray body 115 for storage or transportation before use, as shown in FIGS. 16, 17, and 31. The securing attachment elements 150 can include holes or pinch points, as seen in FIGS. 1-3 and 24-31. The tubing 170 can also itself be used to secure itself to the tray body 115. For example, tubing 170 attached to the fill needle 190 can have its excess length wound around the tray body 115 and fill needle 190, and its loose end can be secured to itself using a tubing connector 175 as seen in FIGS. 23A-23B.

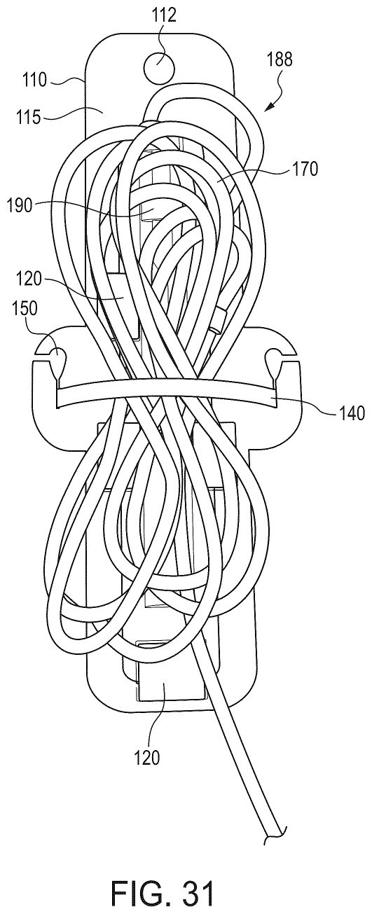

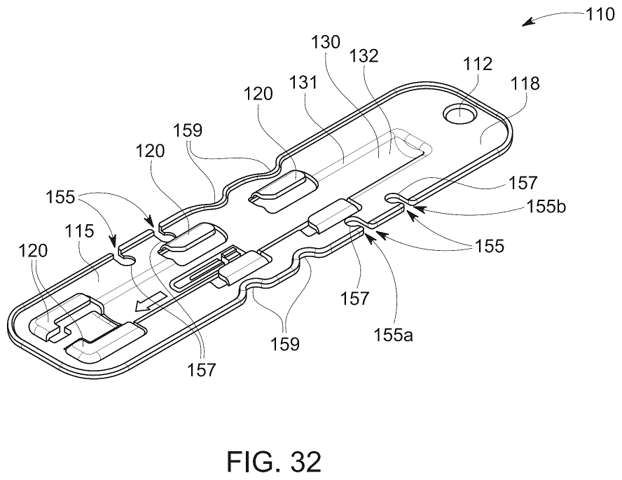

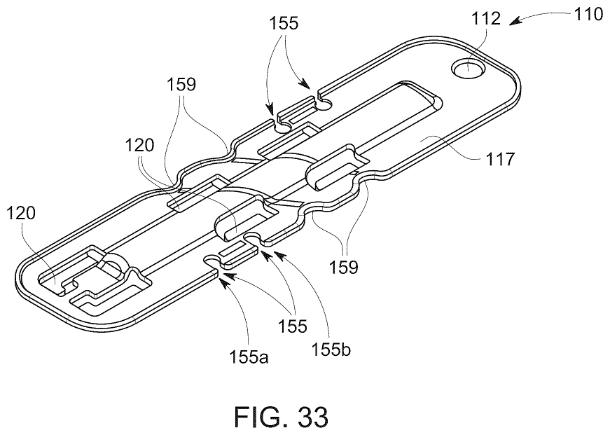

As shown in FIGS. 32 and 33, some embodiments of the tray assembly 110 can include at least one securing notch 155 that is configured to secure an embodiment of the securing mechanism 140 (such as shown in FIG. 31). For example, the securing mechanism 140 can include a separate tubing, cord, flexible elongated material, etc. The securing mechanism 140 can be configured to releasably secure tubing 170 (FIG. 31) of an embodiment of the needle assembly 188 (FIG. 31). For example, the securing mechanism 140 can keep such tubing 170 untangled, protected, and organized, as illustrated and discussed herein. For example, the securing mechanism 140 can include a piece of tubing that is separate from the tubing 170 of the needle assembly.

Each securing notch 155 can include an opening along a side of the tray body 115, and the opening can include and/or extend to a cutout having a circumference 157 that is sized to capture a part of the securing mechanism 140 (FIG. 31). As shown in FIGS. 32 and 33, at least one side of the tray body 115 can include a pair of securing notches 155, which can allow an embodiment of the securing mechanism to extend through a first securing notch 155a and a second securing notch 155b by extending between the first and second securing notches 155a, 155b. For example, the securing mechanism 140 can form a friction and/or compression fit within each of the first and second securing notches 115a, 155b, thereby securing a part of the securing mechanism to the tray body 115. As shown in FIGS. 32 and 33, both of the elongated sides of the tray body 115 can include a pair of securing notches 155 which can allow, for example, opposing ends of an embodiment of the securing mechanism 140 to be efficiently and effectively secured to the tray body 115. For example, securing of a part of the securing mechanism 140 to a securing notch 115 can include pushing the securing mechanism through the opening and against the circumference 157 of the securing notch 115. Additionally, the securing notches 155 can allow for efficient and effective release of the securing mechanism 140 from the tray body 115 thereby allowing for efficient release (e.g., time efficient and/or user-efficient, such as single-handed release) of the securing mechanism 140 from the tray body 115. For example, releasing of a part of the securing mechanism 140 from the securing notch 115 can include pulling the securing mechanism 140 away from the circumference 157 and out the opening of the securing notch 115.

As shown in FIGS. 32 and 33, the elongated sides of the tray body 115 can include at least one curved indent 159 that is positioned adjacent at least one securing notch 155 and is shaped to capture at least a part of the securing mechanism 140 (FIG. 31). The curved indent 159 can assist with securing a position of the securing mechanism 140 along the associated elongated side of the tray body 115. The curved indent 159 can assist with securing the securing mechanism 140 in place to thereby assist the securing mechanism 140 with securing the tubing 170 (FIG. 31) of the needle assembly 188 (FIG. 31) to the tray body 115.

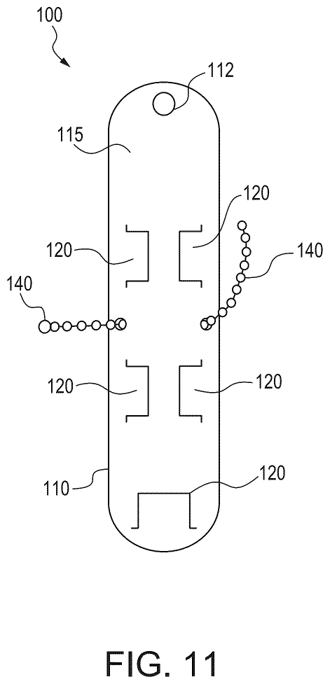

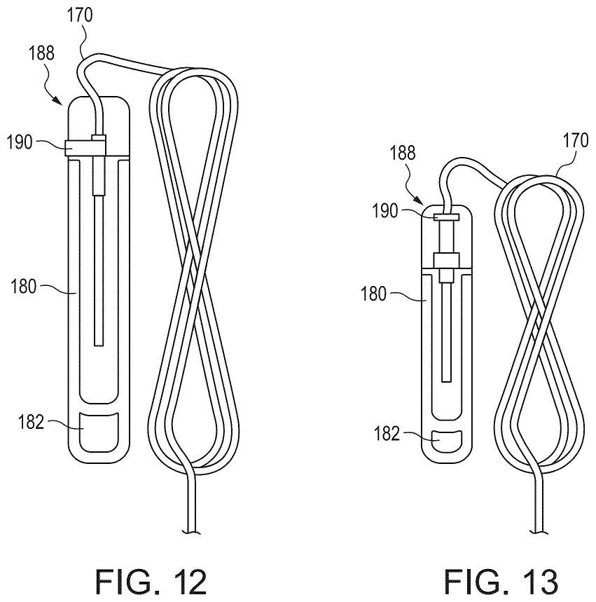

A needle and tubing organizing support kit can include a tray assembly 110, a fill needle 190, a sheath 180, tubing 170, and a securing mechanism 140. As shown in FIGS. 11-17 and 28-31, a needle and sheath organizing support kit can be assembled by first providing the tray assembly 110. The fill needle 190 can be placed within the sheath 180, and the sheath 180 (containing fill needle 190) can be placed into the capturing recess 130 of the tray assembly 110 by sliding the sheath 180 down into the capturing recess 130 and sliding portions of sheath 180 into the sheath receiving slots 138 defined by the retention elements 120 and the tray body 115. Tubing 170 connected to the fill needle 190 can be arranged into a bundle, which can then be placed over the fill needle 190 and sheath 180 on the tray assembly 110. The securing mechanism 140 can then be actuated to retain the sheath 180 and fill needle 190 at least partially within the capturing recess 130 and secure the bundled tubing 170 against the tray body 115 for organization and management of tubing. The assembled kit can then be placed within a sterile bag for transfer into an isolator. An end user in the fill and finish process can set up the needle and tubing organizing support kit within the isolator in approximately backwards order of the steps above. The needle and tubing organizing support kit can be placed into an isolator using the sterile bag and removed from the sterile bag. The user can then manipulate the securing mechanism 140, using only one hand if desired, to gain access to the bundled tubing 170. The user can manipulate the bundled tubing 170 to remove it from the tray body 115 and gain access to the sheath 180 containing fill needle 190. The user can separate the sheath 180 from the tray assembly 110 by sliding the sheath 180 upwards and out from the sheath receiving slots 138 formed by the retention elements 120 and the tray body 115. The user can then remove the fill needle 190 from the sheath 180, and be ready to start the fill and finish process.

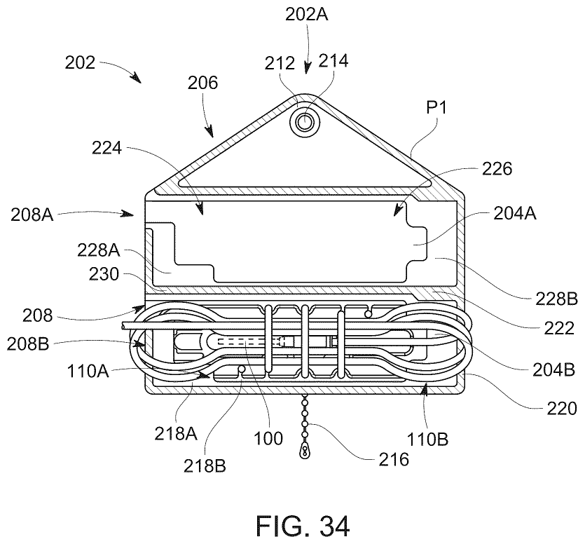

FIG. 34 illustrates a line set wrap 202 in an open configuration 202A and configured to receive the needle and tubing organizing support system 100, including the tray assembly 110, the needle assembly 188 with a portion of the needle 190 secured in a needle sheath 180, and the tubing 170 of the needle assembly secured to the tray assembly 110. In an example, the line set wrap 202 includes two pouches 204A, 204B to receive two needle and tubing organizing support systems 100. In other examples, the line set wrap 202 can include more or less than two pouches to receive the corresponding number of needle and tubing organizing support systems 100.

The line set wrap 202 includes a head portion 206 and a body portion 208. For example, the head portion 206 and the body portion 208 can form one integral part. Alternatively, the head portion 206 can be connected, coupled, attached, or fastened to an upper end 208A of the body portion 208. The head portion 206 can include a mounting element 212 to assist with mounting the line set wrap 202 to a fixture (e.g., hanger, hook, etc.), such as part of a setup step for performing a fill process. In some embodiments, the mounting element 212 can include a hole or aperture or opening 214 through the head portion 206. Other embodiments of the mounting element 212 can be included in the line set wrap, such as one or more of a hook, a hook and loop fabric, a magnet, and the like. The body portion 208 can include the two pouches 204A and 204B, as shown in FIG. 34, and configured to receive the one or more needle and tubing organizing support systems 100. Additionally, a lower end 208B of the body portion can be coupled to a line set wrap securing element 216. In an example the line set wrap securing element 216 can be a beaded cable or beaded tie.

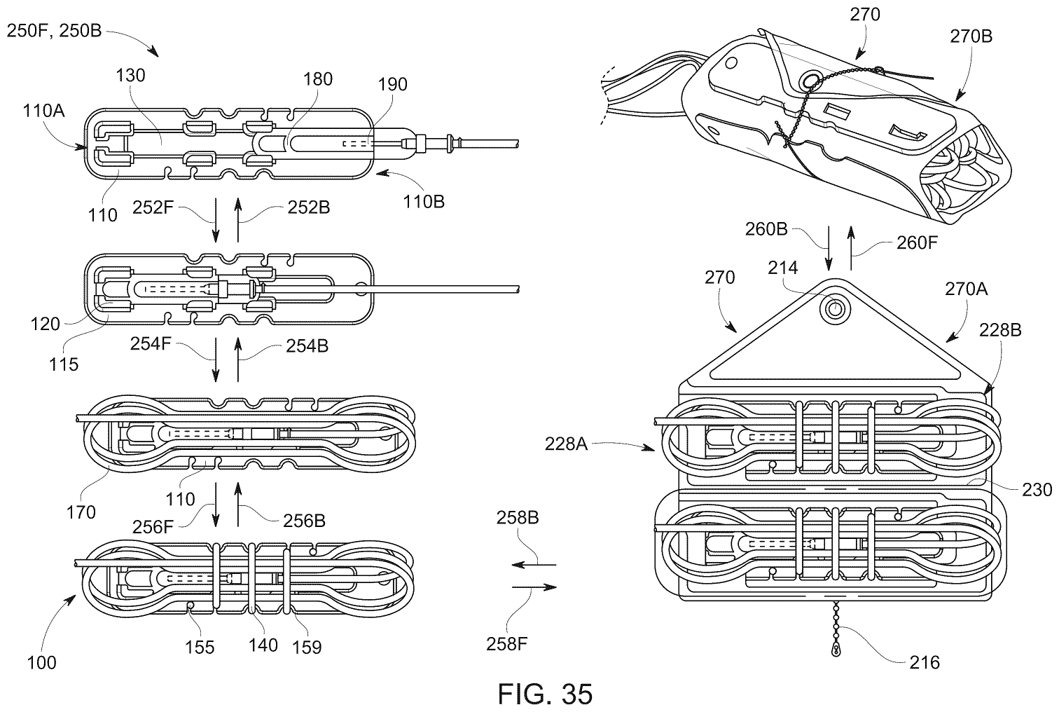

The line set wrap 202 can be sized to fit varied dimensions of the needle and tubing organizing support systems 100. For example, the line set wrap 202 can be made by sealing two sheets of films, a top sheet 218A and bottom sheet 218B, such as Thermo Scientific™ CX3-9 film. Alternatively, a single sheet of Thermo Scientific™ CX3-9 film can be folded onto itself to form the two sheets 218A, 218B, and sealed by film sealing techniques. The line set wrap 202 includes a perimeter seam 220 along a majority of a perimeter Pl of the line set wrap 202 and mid-way seams 222 to delineate the pouches 204A and 204B, as shown in FIGS. 34. Each of the pouches 204A, 204B includes a cavity 224 having an opening 226 bounded by a left side tab 228A and a right-side tab 228B. In other words, the cavity 224 includes the bottom sheet 218B as the bottom with the top sheet 218A trimmed to have the opening 226 bounded by the left side and right-side tabs 228A, 228B. The pouches 204A, 204B are configured to receive the tray assembly 110 in the cavity 224 through the opening 226 with a top portion 110A of the tray assembly secured under the left side tab 228A and a bottom portion 110B of the tray assembly 110 secured under the right-side tab 228B (Also see FIG. 35). Alternatively, the pouches 204A, 204B are configured to receive the tray assembly 110 in the opening with the top portion 110A of the tray assembly 110 secured under the right-side tab 228B and the bottom portion 110B of the tray assembly 110 secured under the left side tab 228A. The midway seams 222 functions as fold lines 230 for the line set wrap 202. Upon receiving the needle and tubing organizing support systems 100 in the two pouches 204A and 204B, the line set wrap 202 can be rolled up or folded up along the fold lines 230 to a folded configuration 270B (see FIG. 35), including stacking up the needle and tubing organizing support systems 100 in the two pouches while securing the line set wrap securing element 216 into the mounting hole 214. The folded configuration 270B of the line set wrap 202 offers a convenient and sterile placing of one or more needle and tubing organizing support systems 100 in a beta bag while performing a fill and finish process.

For example, the line set wrap 202, including two pouches, can have dimensions of length of at least 10.43 inches and width of at least 11.10 inches, whereas the line set wrap including four pouches, can have dimensions of length of at least 17.53 inches and width of at least 11.10 inches. The dimensions of the line set wrap 100 can be configured based on the size of the needle and tubing organizing support systems 100 to be received in the line set wrap 202. The line set wrap 202 can include a triangular shape for the head portion 206, and a rectangular shape for the body portion 208 and/or have one or more of a variety of shapes and sizes for the head and body portions 206, 208. The line set wrap 202 can be made out of one or more materials, such as a polymer like polyester, polyethylene, polypropylene, polymethyl methacrylate, elastomers, a CX3-9 film, tie layer, and nylon. The line set wrap 202 can be made of a material that is not easily punctured by sharp edges, thereby increasing the efficiency of the fill and finish process.

FIG. 35 illustrates forward process 250F, for formation of line set wrap assembly 270 in a closed configuration 270B and backward process steps 250B starting from the closed configuration 270B to the releasing of the needle 190 from the needle sheath 180. Typically, the forward process steps 250F occur outside of an isolator wherein the line set wrap assembly 270 is assembled to include the required number of needle and tubing organizing support systems 100. In comparison, the backward process steps occur inside an isolator wherein the closed configuration 270B of the line set wrap assembly 270 is opened into an open configuration 270A to eventually the needle 190 is released from the needle sheath 180 for performing the filling step of the fill and finish process.

For example, in the forward process 250F, at steps 252F the sheath 180 containing at least a portion of an embodiment of the fill needle 190 (of a needle assembly 188) can be placed into the capturing recess 130 of the tray assembly 110 by sliding the sheath 180 down and along the capturing recess 130 (e.g., from the top of the capturing recess 130) and into and along the sheath receiving slots 138 (e.g., between the tray body 115 and the retention elements 120). At steps 254F and 256F, the tubing 170 connected to the fill needle is gathered around the tray assembly 110 and secured to the tray assembly 110 by the securing mechanism 140 to form the needle and tubing organizing support systems 100, respectively. Notably, the securing mechanism 140 can be captured in the securing notches 155 and curved indents 159 to secure the tubing 170 around the tray assembly 110. At step 258F, a pair of the needle and tubing organizing support systems 100, thus prepared, is secured in the line set wrap 202 to form the open configuration 270A for the line set wrap assembly 270. The line set wrap assembly 270A containing needle and tubing organizing support systems 100 is retained within the cavity by the left-side and right-side tabs 228A, 228B (See FIG. 34) even when it is rotated in all three rotational directions (pitch, yaw, and roll), thus enabling the needle and tubing organizing support system 100 to be a robust solution for fill and finish processes. Finally, at step 26OF the line set wrap 202 is folded up along the fold lines 230 and the line set wrap securing element 216 is secured into the hole 214 to form a closed configuration 270B of the line set wrap assembly 270.

In the backward process 250B, at step 260B, the line set wrap assembly in the closed configuration 270B can be held inside the isolator and the line set wrap securing element 216 can be released from hole 214 to obtain the line wrap assembly 270 in the open configuration 270A. At step 258B the needle and tubing organizing support systems 100 can be released from the line set wrap 202. At step 256B, securing mechanism 140 can be released from the securing notches 155 and curved indents 159 to open up the tray assembly 110 for easy access to the needle sheath 180. At steps 254B and 252B, the needle sheath containing needle 190 is released from the capturing access 130, and needle 190 is pulled out from the sheath 180, respectively. Now, needle 190 is ready for use in a fill-and-finish process.

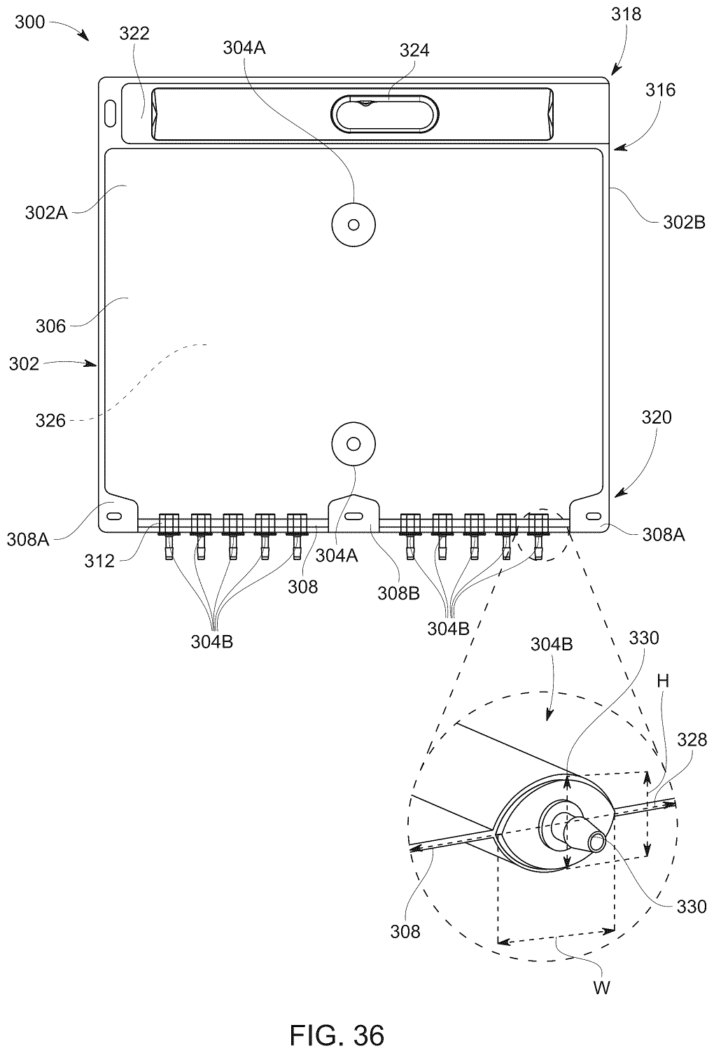

As illustrated in FIG. 36, a surge bag 300 includes a pliable housing 302, and one or more ports 304A, 304B. For example, port 304A can be a face port, while port 304B can be an edge port. In other examples, port 304A can be an inlet port, while port 304B can be an outlet port. For example, the surge bag 300 can include one inlet port 304A and ten outlet ports 304B. In other examples, surge bag can have one inlet port 304A, and one outlet port 304, or any combination of inlet and outlet ports 304A, 304B. The pliable housing 302 is formed from any suitable combination of panels 306 and/or seams 308. For example, in one embodiment the pliable housing 302 includes two panels 302A, 302B secured to each other along a seam 308 to define a cavity configured to receive and hold a fluid, such as a liquid, therein. In another embodiment, the pliable housing 302 includes one panel 306 folded over on itself and secured along the seam 308 to define the cavity. Other embodiments include a plurality of the panels 302 secured to each other along a plurality of seams 308 to define the cavity.

Each of the panels 302 comprises one or more plies of a flexible sheet or film-like material for providing pliability and support to a fluid contained within the cavity. The material includes any pliable, thermoplastic material and may include, by way of example only, ethylene vinyl acetate. Each of the panels can include a 3 layer film, such as Thermo Scientific™ CX3-9 film or CX7-14 film.

The pliable housing 302 can be any shape and/or size as determined by the cavity formed therein. Suitable sizes of the pliable housing 302 include, but are not limited to, greater than about 50 L, between about 50 L and about 200 L, up to about 50 L, between about 100 mL and about 50 L, up to about 25 L, up to about 20 L, between about 100 mL and about 20 L, up to about 16 L, up to about 6 L, about 2 L, between about 100 mL and about 1 L, or any combination, sub-combination, range, or sub-range thereof. Suitable shapes of the pliable housing 302 and/or the cavity include, but are not limited to, spherical, semi-spherical, square, rounded square. To fill the cavity with fluid, a fluid source is connected to the inlet port 304A through tubing 170. To withdraw fluid from the cavity and use it in the fill and finish process, a needle 190 can be connected to the outlet port 304B via tubing 170. The outlet port 304B is coupled to a break 312 in the seam 308, the outlet port 304B and the break 312 together providing access to the cavity within the pliable housing 302. In one embodiment, the ports 304A, 304B provide a sterile inlet to and/or outlet from the cavity within the pliable housing 302. As shown in FIG. 36, the surge bag 300 includes a plurality of ports 304A, 304B. For example, the plurality of ports 304A, 304B can include a male port, a female port, and/or an injection port. Each of the ports 304A, 304B can be used to provide fluid to, or remove fluid from, the cavity within the pliable housing 101. Each of the ports 304A, 304B can additionally include a cap, a clamp, and/or any other feature to facilitate coupling of the port 304A, 304B and/or control of fluid flow into, or out of, the pliable housing 302.

The surge bag 300 includes a body 316 having an upper portion 318 and a lower portion 320. The upper portion 318 includes a tab 322 to display information about the details of the surge bag and a grommet 324 serve as a fastening support. The lower portion 320 includes the two panels 302A and 302B sealed around the perimeter with the seams 308 to encompass an interior cavity 326 to hold a fluid. The lower portion 320 of the surge bag 300 has a pair of corner seams 308A and a middle seam 308B having an extended step-like structure, which facilitates for maximum draining of fluid from the surge bag and reduces fluid hold up in the surge bag during a fill and finish process. Each of the port 304B can be an elliptical port as shown in inset of 304B. The elliptical port 304 B can have a major axis 328 extending along the seam 308 and a minor axis 330 extending orthogonal to the major axis. A width W of the elliptical port 304B can be greater along the major axis 328 than the height H of the elliptical port extending along the minor axis 330. The elliptical port 304B provides for efficient fluid transfer in a sterile manner. As shown in the inset of FIG. 36, outlet port 304B can include a barbed stem 330 for connection of a tube or other external device

In some embodiments, ports 304B of surge bag can include luer ports, tri-clamp ports, quick connect, disconnect ports, barbed ports, needle-free ports, screw cap ports, pinch clamp ports, weldable ports, boat-shaped ports, spike ports, septum ports or custom ports configured for efficient fluid transfer from the surge bag 300.

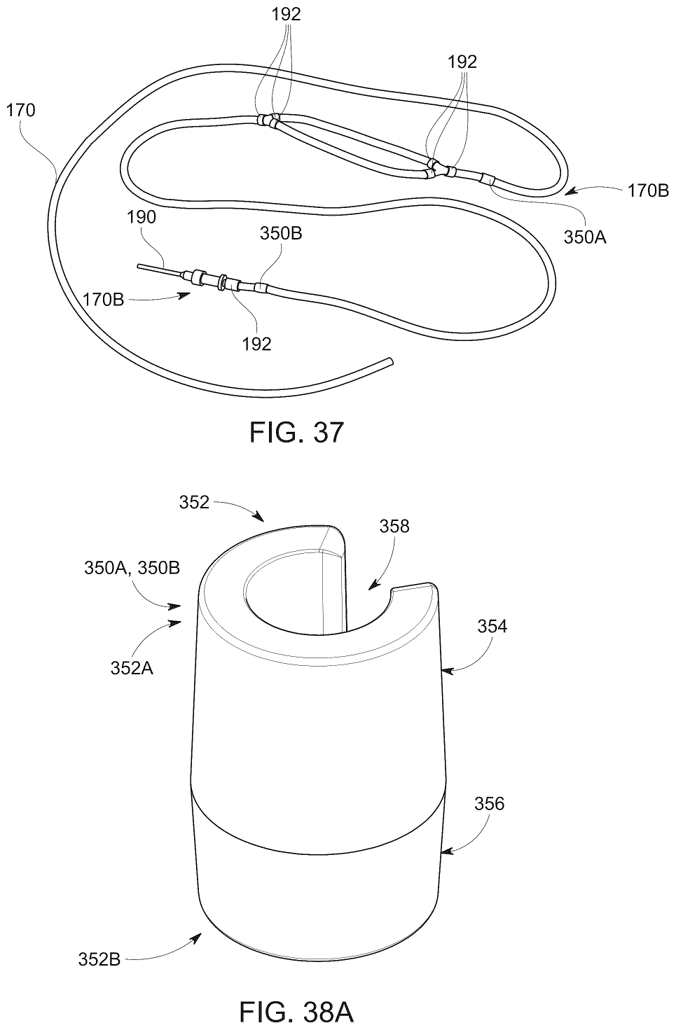

The tubing 170 as shown in FIG. 37 can be attached to each of the outlet ports 304B of the surge bag 300 to withdraw fluid and perform the fill and finish process. The surge bag shown in FIG. 36 includes ten ports and identifying the connection between the tubing 170 and the corresponding port from which the tubing is extending can be ambiguous over the length of the tubing 170. First and second line identifiers 350A, 350B secured to the tubing at a first end 170A and second end 170B of the tubing 170 can help associate the tubing to the corresponding port 304B on the surge bag 300. Any number of additional line identifiers can be coupled to the tubing 170 based on a length of the tubing. Line identifiers 350A, and 350B secured to the tubing 170 extending from a first outlet port 304B can have a common color or numeral printed or engraved on them to distinguish from tubing extending from adjacent ports. For example, for the tubing 170 extending from the ten ports 304B of the surge bag 300, the line identifiers can have ten colors or consecutive numbers from 1 to 10 printed on them. Thus, tubing 170 extending from adjacent ports 304B has line identifiers 350A, 350B differing in color or numerals.

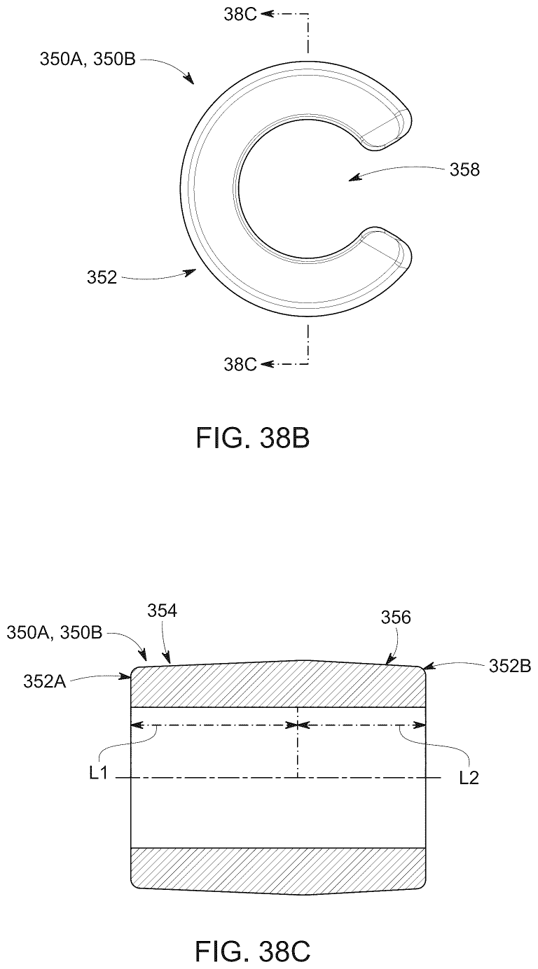

The line identifiers 350A, 350B are shown in FIG. 38A, FIG. 38B, and FIG. 38C in a perspective, top view, and cross-sectional view. The line identifiers 350A, 350B have an open cylindrical wall structure 352, including a first end 352A, and a second end 352B. Further, the cylindrical wall structure 352 includes an upper cylindrical portion 354 and a lower cylindrical portion 356. For example, the upper cylindrical portion 354 and the lower cylindrical portion 356 can form one integral part. Alternatively, the upper cylindrical portion 354 can be connected, coupled, attached, or fastened to the lower cylindrical portion 356. The upper and lower cylindrical portions 354, 356 can include a common opening 358 configured to receive tubing 170. The line identifiers 354A, 354B, and their respective openings 358 can be of a variety of sizes and dimensions to fit tubing 170 of various outer diameters including, but not limited to, ¼ inches, 5/16 inches, or 7/16 inches. In an example, the line identifiers 350A and 350B can be made as an injection molded part composed of either high-density polyethylene (HDPE), high-density polypropylene (HDPP) or any other suitable polymeric material. As shown in FIG. 38B line identifiers 350A, and 350B include a C-shaped cross-section. However, other shapes and cross sections configured to grip tubing 170 fall under the scope of this disclosure.

In some embodiments, as shown in FIG. 38C, upper portion of line identifiers 350A, 350B have a length L1 greater than a length L2 of the lower portion 356. In other embodiments, length L1 of the upper portion 354 can be equal to or lesser than the length L2 of the lower portion 356. Also, the wall thickness of the upper portion 354 increases continuously from the first end 352A as it reaches the lower portion 356. In comparison, the wall thickness of the lower portion 356 decreases as it reaches the second end 352B, giving an overall barrel shape. In other embodiments, the wall thickness of the upper portion and lower portions 354, 356 can remain equal from the first end 352A to the second end 352B. In yet other embodiments, the wall thickness of the upper portion can first decrease from the first end 352A until the lower portion 356, and the wall thickness of the lower portion 356 can increase towards the second end 352B.

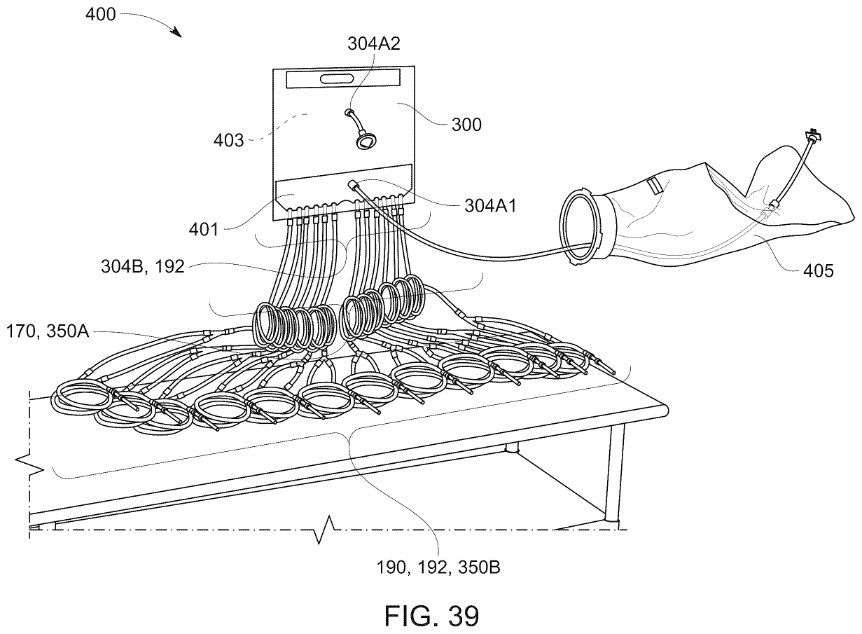

FIG. 39 illustrates a fill and finish system 400, including the surge bag 300 with first face port 304A1 functioning as an inlet port, second face port 304A2 functioning as a vent port, ten outlet ports 304B and fluid 401 contained inside the cavity 403 of the surge bag 300 for delivering to receiving containers (not shown in FIG. 38). Each of the ten outlet ports 304B is connected to their respective needles 190 via the tubing 170. A plurality of compression collars 192 secure the connection between the surge bag 300 and tubing 170 at ports 304B and the connection between the tubing 170 and the needles 190. Tubing 170 extending from adjacent ports is fitted with line identifiers 350A, 350B that help identify the port 304B and its corresponding needle connection 190. The fill and finish system 400 also includes a beta bag 405, which minimizes contamination risk during the transfer of sterile filling assembly, including surge bag 300 connected to needles 190 via tubing 170 into an isolator environment (not shown in FIG. 38).

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, in some implementations, the phrase “approximately” may include +/−0.03 mm, +/−0.05 mm, or the like.

1-80. (canceled)

81. A bag for a fill and finish assembly, comprising:
a pliable housing comprising at least one panel and a perimeter seam defining a cavity within the pliable housing;
at least one inlet port extending from the pliable housing;
at least one outlet port extending from the pliable housing; and
at least one corner seam extending from the perimeter seam.

82. The bag of claim 81, wherein the at least one corner seam comprises a stepped structure.

83. The bag of claim 81, wherein the at least one inlet port comprises a face port.

84. The bag of claim 81, wherein the at least one outlet port comprises an edge port.

85. The bag of claim 81, wherein the at least one inlet port is connected to a fluid source by a tubing.

86. The bag of claim 81, wherein the at least one outlet port is connected to a needle assembly by a tubing.

87. The bag of claim 85, further comprising a compression collar secured proximate to the at least one inlet port.

88. The bag of claim 86, further comprising a compression collar secured proximate to the at least one outlet port.

89. The bag of claim 81, wherein at least one outlet port comprises an elliptical port.

90. The bag of claim 89, wherein the elliptical port comprises a major axis and a minor axis, the major axis extending along the seam and the minor axis extending orthogonal to the major axis.

91. The bag of claim 89, wherein the elliptical port comprises a barbed stem.

92. A line set wrap for a fill and finish assembly, comprising:
a head portion; and
a body portion comprising at least two pouches, wherein each pouch is configured to receive a needle and tubing organizing support system, and
the head portion is coupled to an upper end of the body portion.

93.-94. (canceled)

95. The line set wrap of claim 92, wherein the body portion comprises a perimeter seam and a midway seam.

96. The line set wrap of claim 95, wherein the midway seam forms a fold line for folding the line set wrap.

97. A fill and finish assembly, comprising;
a surge bag comprising at least one inlet port and one outlet port;
a needle assembly connected to the outlet port by a tubing; and
a first line identifier secured to the tubing.

98. The fill and finish assembly of claim 97, further comprising a second line identifier secured to the tubing.

99. The fill and finish assembly of claim 98, wherein the first and second line identifiers have a same color or an identical reference numeral printed.

100. The fill and finish assembly of claim 99, wherein the line identifiers comprise a body having a cylindrical wall structure.

101. The fill and finish assembly of claim 100, wherein the cylindrical wall structure comprises an opening in the wall structure configured to grip onto the tubing.

102. The fill and finish assembly of claim 100, wherein the line identifiers are configured to secure onto tubing having an outer diameter of ¼ inches, 5/16 inches, or 7/16 inches.
